(19) United States
(12) Patent Application Publication
(10) Pub. No.: US 2024/0268749 A1
(43) Pub. Date: Aug. 15, 2024

(54) SMART UNDERWEAR SYSTEMS AND METHODS FOR DETECTING AND MANAGING GASTROINTESTINAL DISORDERS

(71) Applicant: UNIVERSITY OF MARYLAND, COLLEGE PARK, College Park, MD (US)

(72) Inventors: Brantley HALL, Bethesda, MD (US); Santiago BOTASINI, Hyattsville, MD (US); Reza GHODSSI, Potomac, MD (US)

(21) Appl. No.: 18/568,750

(22) PCT Filed: Jun. 8, 2022
(86) PCT No.: PCT/US2022/032622

§ 371 Date: Dec. 8, 2023

Related U.S. Application Data
(60) Provisional application No. 63/209,959, filed on Jun. 11, 2021.

(51) Int. Cl. A61B 5/00; A61B 5/1455; A61B 5/1477

(52) U.S. Cl. CPC A61B 5/4255; A61B 5/1455; A61B 5/1477; A61B 5/6804

(57) ABSTRACT

A system for detecting a gastrointestinal disorder includes a first sensor configured to generate a first signal indicative of a sensing of a first concentration of a biomarker in a flatus, a second sensor configured to generate a second signal indicative of a sensing of a second concentration of the biomarker in the flatus, a filter disposed on the second sensor, a processor, and a memory. The filter is configured to selectively remove the biomarker from the flatus prior to sensing by the second sensor. The memory includes instructions stored thereon, which when executed by the processor, cause the system to compare the first signal to the second signal to determine a concentration of the biomarker and provide an indication of the concentration of the biomarker, based on the comparison.

### CROSS-REFERENCE TO RELATED APPLICATION/CLAIM OF PRIORITY

This application claims the benefit of, and priority to, U.S. Provisional Patent Application No. 63/209,959, filed on Jun. 11, 2021, the entire contents of which are hereby incorporated herein by reference.

### TECHNICAL FIELD

The present disclosure relates generally to the field of detecting, managing, and informing the treatment of gastrointestinal disorders. More specifically, an aspect of the present disclosure provides systems and methods for measuring biomarkers produced by the human gut microbiota for detecting, managing, and treating gastrointestinal disorders, such as Irritable Bowel Syndrome (IBS) and Inflammatory Bowel Disease (IBD) and its primary clinical manifestations: Crohn's disease and ulcerative colitis.

### BACKGROUND

Irritable Bowel Syndrome (IBS) is a widespread gastrointestinal disorder. It is characterized by disruptions in typical gastrointestinal function resulting in severe gas, bloating, diarrhea and/or constipation. IBS imposes a substantial cost on both patients and the healthcare system and is highly associated with depression and anxiety. The next major step in IBS management and treatment will be the division of IBS into subtypes based on the underlying cause. One of these underlying causes is Small Intestine Bacterial Overgrowth (SIBO), the increased abundance of microbes in the small intestine, which leads to excessive production of the gas hydrogen due to bacterial fermentation of readily abundant carbohydrates. Excessive hydrogen production in the small intestine is hypothesized to cause the major symptoms of bloating and flatulence that can go from a mild discomfort to a severe pain, affecting the quality of life. In some cases, methanogenic archaea can overgrow leading to excessive production of the gas methane which is associated with constipation.

The main symptom of hydrogen dominant SIBO is bloating, abdominal pain, excessive flatulence and diarrhea. Many individuals with IBS have SIBO. Doctors mostly diagnose IBS by an elimination process of other possible diseases and mostly rely on patient symptomatology and medical history. The gold-standard diagnostic test for SIBO requires an endoscopy, however due to its invasiveness and expense, an endoscopy is rarely performed for SIBO. The next closest analysis is from hydrogen breath testing. However, it has low sensitivity and specificity due to low (˜0-100 ppm) concentrations of microbial-produced gases in breath. Additionally, the test is time-consuming and labor-intensive. Moreover, even when the doctor asserts his diagnosis, there is no objective way to follow-up the intervention treatment rather than relying on the patient's perception. Therefore, there is an urgent need for improved tests specially for SIBO where in some severe cases requires the prescription of antibiotics.

Inflammatory Bowel Disease (IBD) is a relapsing-remitting inflammatory disease of the gastrointestinal tract. There are two major clinical manifestations of IBD: Ulcerative Colitis and Crohn's disease. IBD is a debilitating life-long disease. The goal of IBD treatments is to induce a temporary resolution of inflammation called remission. However, due to lifestyle and dietary factors, most patients frequently have periods of severe inflammation, called flares. Finding effective treatment plans that induce and maintain remission is difficult due to inter-individual differences in the response to drugs. IBD patients are prescribed a series of increasingly potent anti-inflammatory drugs while IBD disease activity is monitored. IBD treatments are far from perfect, most patients still experience flares. IBD is a chronic disease and places a major burden on the healthcare system.

A major impediment to IBD treatment is the lack of tools to measure non-invasive biomarkers of intestinal inflammation. Endoscopic evaluation of inflammation is the gold standard for IBD diagnosis. However, endoscopies cannot be performed frequently enough to keep pace with changes in IBD disease activity. Therefore, there is an urgent need for new non-invasive biomarkers to assess IBD disease activity to inform the patient and doctors to act before the symptoms arrive.

IBD disease activity is associated with increases in the concentrations of nitric oxide (NO), nitrogen dioxide (NO₂) and hydrogen sulfide (H₂S) in gaseous rectal effluent (flatus). NO rapidly reacts with Oxygen (O₂) to produce NO₂. Previous studies using endoscopy have observed far higher luminal concentrations of NO₂ in IBD patients compared to controls. Likewise, an excessive H₂S production has been hypothesized to be involved in the etiology of Ulcerative Colitis. H₂S has been proposed to predispose individuals to colorectal cancer by promoting low-level inflammation through reduced mucosal integrity or genotoxicity. Therefore, NO₂, NO and H₂S concentrations in flatus can be used to track the severity of intestinal inflammation. This gives the means to make a distinction between IBS and IBD conditions as NO and NO₂ are not presented in IBS patients.

Accordingly, there is interest in detecting gastrointestinal disorders.

### SUMMARY

An aspect of the present disclosure provides a system for detecting and/or managing a gastrointestinal disorder. The system includes a first sensor configured to generate a first signal indicative of a first concentration of a biomarker in a flatus, a second sensor configured to generate a second signal indicative of a second concentration of the biomarker in the flatus, a filter disposed on the second sensor, a processor, and a memory The filter is configured to selectively remove the biomarker from the flatus prior to sensing by the second sensor. The memory includes instructions stored thereon, which when executed by the processor, cause the system to compare the first signal to the second signal to determine a concentration of the biomarker and provide an indication of the concentration of the biomarker, based on the comparison.

In an aspect of the present disclosure, the system may further include a third sensor. The third sensor may be configured to generate a third signal indicative of sensing of a temperature or a humidity.

In another aspect of the present disclosure, the instructions, when executed by the processor, may further cause the system to compensate the first signal and the second signal based on the third signal.

In yet another aspect of the present disclosure, the first sensor and the second sensor may be the same type of sensor.

In accordance with further aspects of the present disclosure, the first sensor and the second sensor may include an electrochemical sensor and/or a metal oxide sensor.

In an aspect of the present disclosure, the system may further include an accelerometer configured to detect motion.

In another aspect of the present disclosure, the instructions, when executed by the processor, may further cause the system to determine whether the system is being worn by a user based on the detected motion.

In yet another aspect of the present disclosure, the system may further include a spectroscopic sensor configured for sensing a color change indicating biomarker detection.

In yet another aspect of the present disclosure, the instructions, when executed by the processor, further cause the system to detect a color change in a Griess reagent, and determine a concentration of the biomarker, based on the color change.

In aspects, the instructions, when executed by the processor, may further cause the system to diagnose the presence or absence of intestinal inflammation based on the indication of the concentration of the biomarker.

An aspect of the present disclosure provides a computer-implemented method for detecting and/or managing a gastrointestinal disorder. The method includes accessing a first signal indicative of a first concentration of a biomarker in a flatus, accessing a second signal indicative of a second concentration of the biomarker in the flatus, comparing the first signal to the second signal to determine a concentration of the biomarker, and providing an indication of the concentration of the biomarker, based on the comparison.

In accordance with further aspects of the present disclosure, the method may further include diagnosing the presence or absence of intestinal inflammation based on the indication of the concentration of the biomarker.

In yet a further aspect of the present disclosure, the first signal may be sensed by a first sensor and the second signal may be sensed by a second sensor.

In yet a further aspect of the present disclosure, the first sensor and the second sensor may be the same type of sensor.

In another aspect of the present disclosure, the method may further include selectively removing the biomarker from the flatus, prior to sensing by the second sensor, by a filter disposed on the second sensor.

In yet another aspect of the present disclosure, the method may further include sensing by a third sensor a third signal indicative of a sensing a temperature or a humidity, and determining whether a system that includes the first sensor, the second sensor and the third sensor is being worn by a user, based on the third signal.

In a further aspect of the present disclosure, the first sensor and the second sensor may include an electrochemical sensor and/or a metal oxide sensor.

In yet a further aspect of the present disclosure, the method may further include detecting motion by an accelerometer.

In yet a further aspect of the present disclosure, the method may further include determining whether a system that includes the first sensor, the second sensor and the accelerometer is being worn by a user, based on the detected motion.

In an aspect of the present disclosure, the method may further include sensing a color change of a Griess reagent disposed in a flatus by a spectroscopic sensor. The color change may indicate biomarker detection.

In an aspect of the present disclosure, the method may further include determining a concentration of the biomarker, based on the color change.

An aspect of the present disclosure provides a smart underwear system for detecting and/or managing gastrointestinal disorder. The system includes an undergarment configured for being worn by a user and a device attached to the undergarment. The device includes a first sensor configured to generate a first signal indicative of a first concentration of a biomarker in a flatus, a second sensor configured to generate a second signal indicative of a second concentration of the biomarker in the flatus, a filter disposed on the second sensor, a processor and a memory. The filter is configured to selectively remove the biomarker from the flatus prior to sensing by the second sensor. The memory includes instructions stored thereon, which, when executed by the processor, cause the device to compare the first signal to the second signal to determine a concentration of the biomarker and provide an indication of the concentration of the biomarker, based on the comparison.

Further details and aspects of the present disclosure are described in more detail below with reference to the appended drawings.

### BRIEF DESCRIPTION OF THE DRAWINGS

A better understanding of the features and advantages of the present disclosure will be obtained by reference to the following detailed description that sets forth illustrative aspects, in which the principles of the present disclosure are utilized, and the accompanying drawings of which:

FIG. 1 is a diagram of an exemplary system for detecting and/or managing gastrointestinal disorders, in accordance with examples of the present disclosure;

FIG. 2 is a block diagram of a controller configured for use with the system for detecting and/or managing gastrointestinal disorders of FIG. 1, in accordance with aspects of the disclosure;

FIG. 3 is a top view of the system of FIG. 1, in accordance with aspects of the present disclosure;

FIG. 4 is a top perspective view of the system of FIG. 1, in accordance with aspects of the present disclosure;

FIG. 5 is a block diagram of the system of FIG. 1, in accordance with aspects of the present disclosure;

FIG. 6 is a diagram illustrating the expression of the inducible nitric oxide (NO) synthase gene NOS2 (Nitric Oxide Synthase 2);

FIG. 7 is a graph illustrating data from a user wearing the system of FIG. 1, in accordance with aspects of the present disclosure;

FIG. 8 illustrates a zoomed-in view of a single flatus from FIG. 7, in accordance with aspects of the present disclosure;

FIG. 9 is a graph illustrating a concentration of compounds in flatus, in accordance with aspects of the present disclosure;

FIG. 10 is a graph illustrating a concentration of rectal NO in flatus based on IBD status, in accordance with aspects of the present disclosure;

FIG. 11 is a graph that shows a comparison between the filtered sensor and the unfiltered sensor with increasing additions of H₂S and a fixed concentration of H₂ using a flatus simulator, in accordance with aspects of the present disclosure;

FIG. 12 shows a graph illustrating calibration curves from the unfiltered sensor by keeping the concentration of H₂ constant for each curve and increasing only the concentration of H₂S, using the flatus simulator, in accordance with aspects of the present disclosure;

FIG. 13 is a graph that shows a calibration curve for hydrogen concentration using the reading from the filtered sensor of FIG. 3, in accordance with aspects of the present disclosure;

FIG. 14 is a graph that illustrates NO₂ detection using the Griess reaction, in accordance with aspects of the present disclosure;

FIG. 15 is a graph that illustrates a linear profile with increasing concentration of NO₂ using a flatus simulator, in accordance with aspects of the present disclosure;

FIG. 16 is a graph that illustrates the stability of the Griess solution, in accordance with aspects of the present disclosure;

FIG. 17 is a graph that illustrates a spectra profile of Griess reaction with nitrite, in accordance with aspects of the present disclosure;

FIG. 18 is a graph that illustrates a calibration curve using the spectrophotometer and Griess reagent with increasing concentration of nitrite, in accordance with aspects of the present disclosure;

FIG. 19 is a graph that illustrates a calibration curve with the spectroscopic sensor 340 of FIG. 3 and 100 μL of Griess reagent with increasing additions of nitrite, in accordance with aspects of the present disclosure; and

FIG. 20 is a flow diagram for a method for diagnosing the presence or absence of intestinal inflammation, in accordance with aspects of the present disclosure.

### DETAILED DESCRIPTION

The present disclosure relates generally to the field of detecting gastrointestinal disorders. More specifically, an aspect of the present disclosure provides systems and methods for measuring biomarkers produced by the human gut microbiota for detecting and/or managing gastrointestinal disorders.

Aspects of the present disclosure are described in detail with reference to the drawings wherein like reference numerals identify similar or identical elements.

Although the present disclosure will be described in terms of specific aspects and examples, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary aspects illustrated in the drawings, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

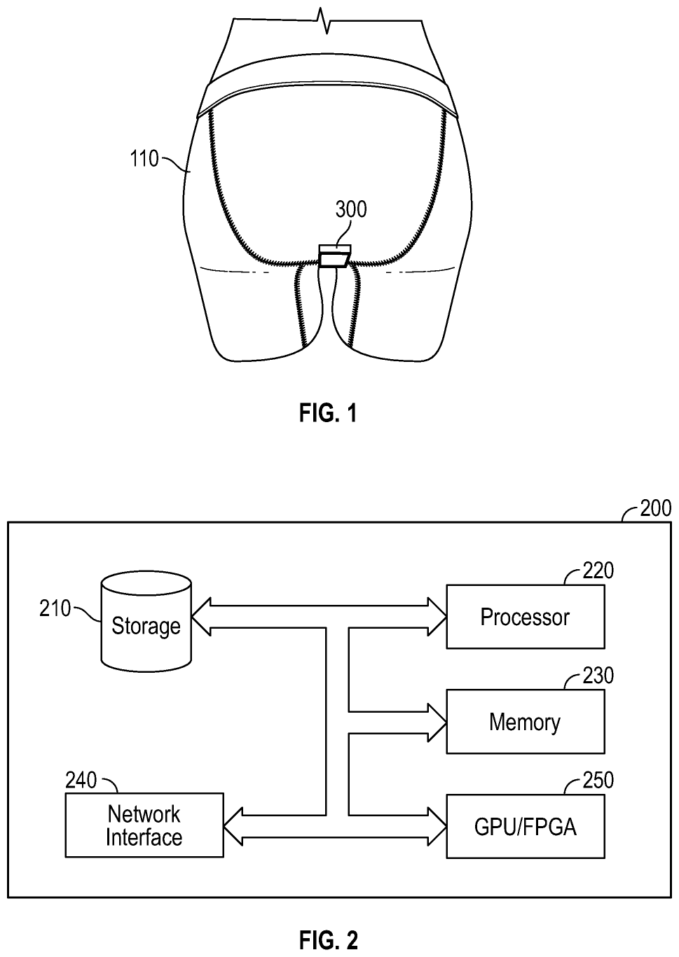

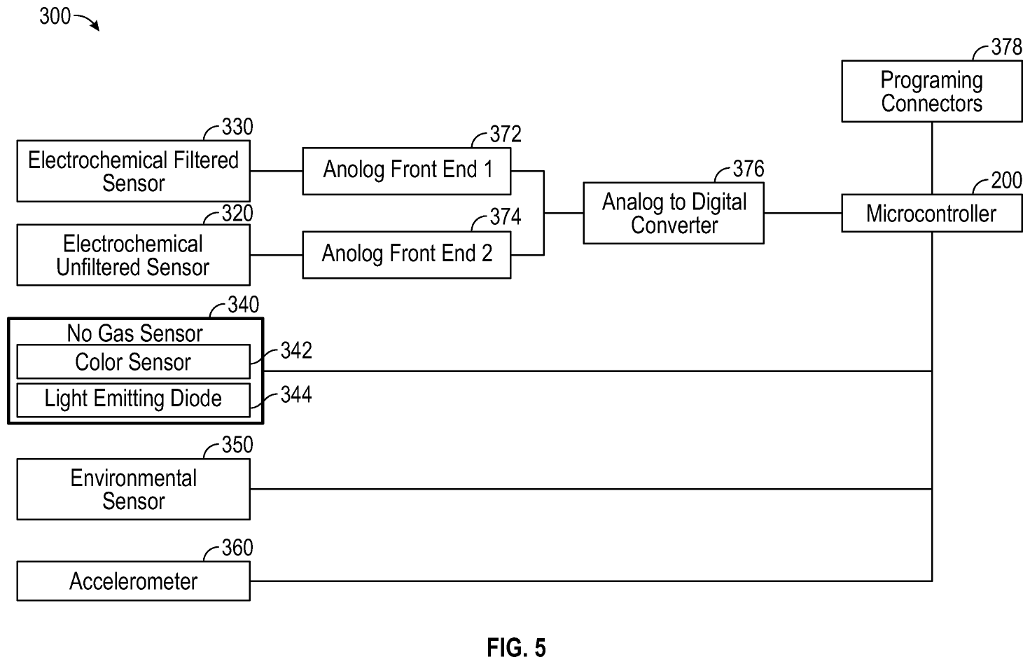

Referring to FIGS. 1 and 3-5, a system 300 for detecting and/or managing gastrointestinal disorders is shown. The system 300 is configured to capture the day-to-day symptoms of gastrointestinal disorders such as Inflammatory Bowel Disease (IBD) and Irritable Bowel Syndrome (IBS) by measuring gas concentration in gaseous rectal effluent (flatus), whose mutual composition acts as biomarkers of the mentioned gastrointestinal (GI) disorders. The system 300 is configured to autonomously measure the frequency, volume, and gas composition of flatus.

The system 300 generally includes a first sensor 320 configured to generate a first signal indicative of sensing a first concentration of a biomarker in the flatus, a second sensor 330 configured to generate a second signal indicative of sensing a second concentration of the biomarker in the flatus, a filter 332 disposed on the second sensor 330, and a controller 200. Flatus is generally a mixture of hydrogen sulfide, hydrogen, methane, carbon dioxide, and numerous volatile organic compounds. Biomarkers of gut inflammation may include, for example, H₂S, H₂, CH₄, CO₂, NO, NO₂, and/or other volatile compounds produced by the human gut microbiome. Flatus is a mixture of gases dominated by hydrogen which causes interference for commercial H₂S and NO sensors. The system 100 includes the advantage of enabling filtering strategies to sense these gases in a high concentration hydrogen background.

Bacterial overgrowth in the small intestine leads to an increase in gut microbial hydrogen (H₂) production, which manifests as an increased frequency of volume and gas composition in flatus. Therefore, abnormal concentrations of H₂ are consistent with Small Intestine Bacterial Overgrowth (SIBO). The disclosed systems and methods may also be used to follow the success of treatment interventions by providing an objective analytical measure.

Flatus is an ideal non-invasive source for sampling because they are more frequent (>10-15 per day) than stool and can be measured passively in real-time or near real-time using sensors. In addition, flatus contains volatile molecules that are rare in or quickly disappear from stool samples. Finally, flatus supplements an ample volume of gas to measure with 500-1,500 mL of gas passed per day. In contrast, stool samples are infrequent, require collection and transport of the bio-sample to a lab, and lab analysis results in delayed readings.

Few studies have analyzed the composition of flatus due to the difficulties in sample collection. Flatus is most often dominated by bacterially produced hydrogen (H₂) and carbon dioxide (CO₂), the last two resulting from the fermentation of host-inaccessible carbohydrates (FIG. 9). If a person is colonized by methanogenic archaea, H₂ may be replaced by methane (CH₄) as the dominant gas in flatus. Other volatiles are present in moderate (>1 ppm) concentrations, including hydrogen sulfide (H₂S) and ammonia. In addition to microbially-produced molecules, host-derived molecules are also present in flatus.

There are likely hundreds of low abundance (<1 ppm) volatile organic compounds (VOCs) present in flatus that have not been previously measured due to the usage of insufficiently sensitive techniques. In fact, the major studies on the composition of flatus were performed more than 20 years ago before recent advances in mass spectrometry enabled the measurement of these low-abundance VOCs. Analysis of the headspace of stool samples has identified hundreds of VOCs present. Studies comparing the VOC profiles of healthy and IBD individuals have noted specific differences and demonstrated a moderate ability to discriminate between disease and health based on the VOC profile. Further analysis of VOCs in flatus could help identify non-invasive biomarkers to predict the onset and/or progression of IBD.

The system 300 is configured to be attached to an undergarment 110 of a user, e.g., as “Smart Underwear” (FIG. 1). For example, the system 300 may be attached to the undergarment 110 adjacent to the perineum. In aspects, the system 300 may be attached to the undergarment 110 using any suitable means, such as double-sided tape, or sewn into a pocket of the undergarment 110.

The first sensor 320 and the second sensor 330 are configured to quantify the production of volatile compounds produced by the human gut microbiome. The first sensor 320 and the second sensor 330 may include, for example, an electrochemical sensor and/or a metal oxide sensor. The system 300 may use the two sensors 320, 330 (e.g., one filtered and one unfiltered), for a background subtraction approach for the measurement of various biomarkers, (e.g., H₂S). In aspects, the first sensor 320 and the second sensor 330 may be the same type of sensor, for example, both sensors may be electrochemical sensors of the same make and model. The filter 332 is disposed on the second sensor 330 and is configured to selectively remove a compound from the flatus prior to sensing by the second sensor. For example, the filter 332 may be configured to selectively remove H₂S. The filter 332 may include multi-layer filtration that selectively removes volatile compounds, such as H₂S, while allowing the other gases present in flatus to pass unimpeded. To determine the concentration of the biomarker in a flatus via subtractive sensing, the signals between two sensors of the same type are compared (one unfiltered and the other filtered). The calibrated difference in the filtered and unfiltered sensors corresponds to the concentration of the biomarker. A benefit of the disclosed technology is that by combining two types of sensors (filtered and unfiltered), the dynamic range of the sensors may be increased to accurately measure various biomarkers (e.g., H₂S) in most flatus. The filter 332 enables the system 300 to measure H₂S even in a high H₂ concentration environment.

Electrochemical sensors use solid electrolytes and are protected by a sealed close case with a gas-permeable membrane on top. Electrochemical sensors work by having gases diffuse through the gas permeable membrane to be reduced or oxidized at an electrode. The oxidation/reduction measurements enable measurement of the volatile compounds of interest.

Metal Oxide (MOx) sensors that can measure volatile compounds are small, widely available, and inexpensive. However, the response of MOx sensors is not specific to a single volatile compound. Due to the cross-sensitivity to the other gases present in flatus, commercially available MOx sensors cannot be used to quantify H₂S in flatus.

Subtractive sensing enables the measurement of H₂S with commercially available electrochemical sensors and MOx sensors.

In aspects, the system 300 may include an environmental sensor 350, the environmental sensor 350 is configured to generate a signal indicative of a temperature or a humidity. The signal indicating the temperature or humidity may be used by the controller 200 to determine whether the system 300 is being worn by a user based on the third signal. In aspects, the system 300 may include an accelerometer (e.g., an inertial measurement unit) configured to detect motion. The controller 200 may determine whether the system 300 is being worn by a user based on the detected motion. An advantage of the Smart Underwear is autonomy. Using sensor fusion of the output of the accelerometer, as well as temperature and humidity values, the controller 200 can classify whether the device is worn or not. Therefore, the only user intervention required is to adhere or place the Smart Underwear device to underwear. This enables the collection of authentic data over a long period of time with minimal interaction required by the user. In aspects, the controller 200 is configured to wirelessly transmit (e.g., by Bluetooth™ or other wireless protocol) the data to a smartphone application. In aspects, the data may be wirelessly transmitted (securely) to other types of authorized systems/devices, including but not limited to local health monitoring devices, remote health monitoring systems (e.g., cloud-based and perhaps operated by a healthcare provider), and/or a combination of a local health monitoring device that provides health monitoring information to a health monitoring system. The controller 200 may use the temperature and/or humidity values to compensate the first and second sensor signals. In aspects, if the humidity value is above a threshold value, the controller 200 may use a weighted first signal and/or second signal value to compensate the first and/or second sensor signals. For example, if the relative humidity is measured as 70%, the controller may multiply the first signal by a value of about 1.1. In another example, if the temperature is measured as 40 degrees Fahrenheit, then the controller 200 may compensate the first and/or second signal by multiplying the first and/or second signal with a value of about 0.9. The above values are only provided as examples, other values are contemplated by this disclosure.

In aspects, the system 300 may further include a spectroscopic sensor 340. The spectroscopic sensor 340 generally includes a light source 344, a light sensor 342, and a reagent 346 (e.g., a Griess reagent). The spectroscopic sensor 340 may be used for nitric oxide (NO) and NO₂ detection to detect the color change in a Griess reaction. The Griess test is an analytical chemistry test that detects the presence of nitrite ions in the solution. NO has a fast reactivity with oxygen to form nitrogen dioxide (NO₂) with a third-order kinetic reaction with a high constant rate in the order of 10⁶ M⁻² s⁻¹: 2NO+O₂→2NO₂.

The majority of NO expelled in flatus will rapidly react with atmospheric oxygen to form NO₂. While electrochemical and metal oxide sensors exist, all commercial off-the-shelf sensors experience interference from H₂. Because the expected concentration range of NO₂ in flatus is between 0-20 ppm, the signal from hydrogen (up to 30,000 ppm) will almost always dwarf the NO₂ signal. Unfortunately, unlike H₂S, there are no available filters to remove the NO or NO₂ to adopt a background-filtration strategy. Additionally, the sensitive chemiluminescent methods for NO measurement require the generation of ozone, which is dangerous in a wearable device.

In aspects, the Griess method may be used to overcome the interference. The Griess method detects nitrites formed from the fast reaction of NO and/or NO₂ and H₂O. The Griess method is a sensitive and selective method to quantify the concentration of nitrites in solution. The colorimetric test is based on the subsequent reactions between the sulfanilic acid (or a sulfanilamide) and the nitrite in an acid media (usually phosphoric acid), to produce the diazonium salt that then couples with an N-(1-naphthyl)ethylenediamine to form a highly colored (red-pink) compound 4-[(E)-{4-[(2-Aminoethyl)amino]naphthalen-1-yl}diazenyl]-benzene-1-sulfonamide (Azo Dye), with a maximum absorption at 548. The colorimetric test has a wide linear range between 1 and 100p M of nitrite. While some NO will be lost to nitrate, which cannot be measured with the Griess reaction, this fraction is small and constant.

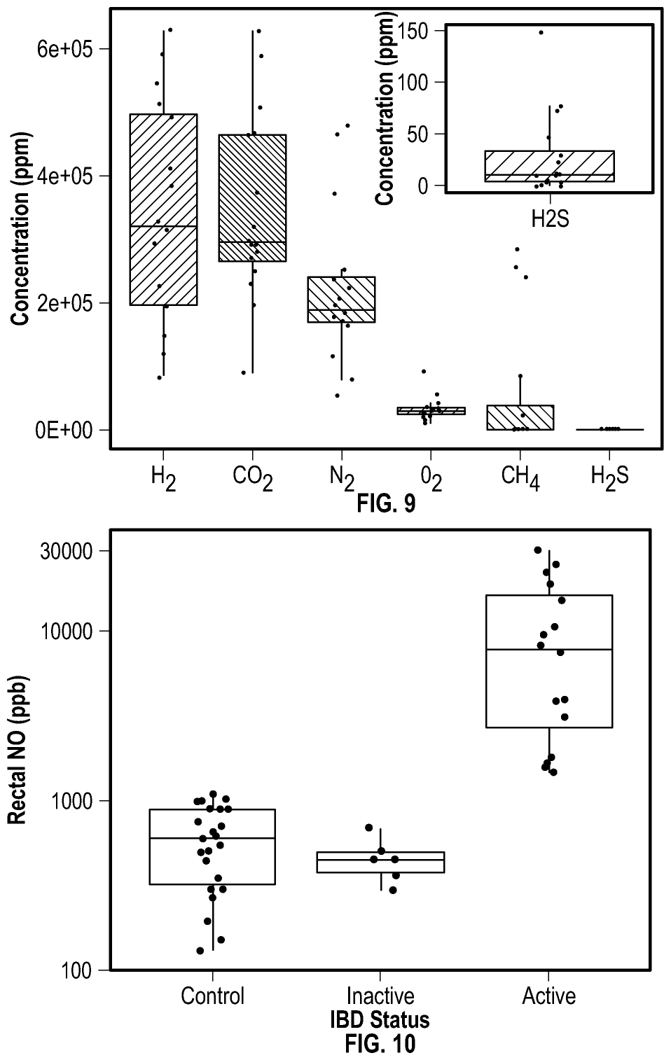

NO concentration may be used as a non-invasive biomarker of Ulcerative Colitis (UC) disease activity. NO, a free radical, is produced by the human enzyme inducible nitric oxide synthase (iNOS) which is encoded by the gene NOS2. NOS2 is expressed in many cell types, including the colonic epithelial cells, allowing the NO to diffuse directly into the lumen. The expression of NOS2, dependent on the transcription factor (NF)-κB, is induced in response to inflammation, and concordantly is strongly upregulated in IBD (FIG. 10). Analysis of rectal biopsies from UC patients in the Human Microbiome Project 2 revealed more than a 12-fold upregulation of expression of NO2 in UC patients compared to controls.

Techniques used to invasively measure NO in either the lumen of the colon or rectum have confirmed a significant increase in the concentration of NO in active IBD. Patients with active UC had NO concentrations above 1,000 ppb with a median of about 7,000 ppb with the maximum concentration observed over 20,000 ppb (FIG. 10). In contrast, all patients with inactive disease had NO concentrations ranging from 300-690 ppb with a median of about 450 bpp. Gas was collected from a catheter inserted 10 cm into the rectum of patients with active UC and into controls. Active UC patients had median NO concentrations of about 10,950 ppb while controls had median NO concentrations of about 154 ppb, corresponding to more than a 70-fold difference in NO concentration. The concentration of NO correlated with the degree of disease activity measured via endoscopy and/or FCP, demonstrating that NO is a useful biomarker for evaluating IBD disease activity.

NO produced in response to inflammation may be expelled in flatus. Due to its presence in flatus, NO is a non-invasive biomarker for IBD disease activity and an indication of flares.

In aspects, hydrogen sulfide (H₂S) in flatus may be a biomarker of UC disease activity. H₂S is a mammalian gasotransmitter with wide-ranging effects on human physiology. Gasotransmitters are gaseous signaling molecules that exert wide-ranging physiological effects on the human body. While low (μM) concentrations of H₂S can play beneficial roles, excessive concentrations (mM) can cause deleterious effects and even be fatal through the inhibition of cytochrome c oxidase. Additionally, excessive H₂S production by the gut microbiota also leads to pungent, malodorous flatulence, which can have negative effects on social and emotional well-being.

In humans, the vast majority of microbially-produced H₂S originates from bacteria in the colon, where luminal concentrations have been estimated to range from about 0.3 mM to about 3.4 mM. H₂S concentrations in the colon are far higher than the threshold for physiological H₂S bioactivity, which is around 100 μM.

Numerous studies support higher H₂S production in IBD. Increased fecal H₂S concentrations have been identified in individuals with IBD. In addition, higher abundances of sulfate-reducing bacteria, which produce H₂S via dissimilatory reduction, have been identified in the stool of UC patients. Bacteria that produce H₂S through the degradation of cysteine have increased in IBD.

In the gastrointestinal tract, excessive H₂S production has been hypothesized to be involved in the etiology of Ulcerative Colitis through several mechanisms. First, increased H₂S production could reduce mucosal barrier integrity by the reduction of disulfide bonds that imbue mucus with its gel-like properties. Second, increased H₂S production could inhibit butyrate oxidation in the colonic epithelia. In the absence of butyrate, colonocytes switch to anaerobic metabolism, which allows unused excess oxygen to diffuse into the colonic lumen. Excessive oxygen in the colonic lumen is then thought to promote the abundance of pathobionts, which could, in turn, produce more H₂S, leading to a positive feedback loop. These studies support additional research into the abundance and roles of H₂S in IBD.

A major impediment to measuring H₂S production is the lack of appropriate tools. Stool samples are a poor bio-sample for H₂S measurements because H₂S rapidly diffuses across the epithelium, where it is detoxified by human enzymes. In addition, H₂S is also highly reactive and therefore introduces time-dependent effects on its measurement in the stool. In total, it is estimated that less than 1% of gut H₂S production is accounted for in stool. Breath testing is an inadequate technique for measuring gut microbial H₂S production. Unlike hydrogen or methane, little gut microbially-produced H₂S reaches the breath due to the short circulating half-life of H₂S arising from active detoxification and its high chemical reactivity. In addition, oral microbes also produce H₂S, which obfuscates the source(s) of H₂S in the breath.

In contrast to stool samples, flatus is an ideal biological sample in which to measure gut microbial H2S production because concentrations are quite high (˜10-150 ppm). Therefore, H₂S concentrations within flatus are well within the sensitivity range of commercially available gas sensors. This allows for an exceptional signal-to-noise ratio. Direct measurement of gut microbial H₂S production in diseases such as IBD is important to determine if H₂S is involved in their etiology.

FIG. 2 illustrates controller 200 includes a processor 220 connected to a computer-readable storage medium or a memory 230. The controller 200 may be used to control and/or execute operations of the system 100. The computer-readable storage medium or memory 230 may be a volatile type of memory, e.g., RAM, or a non-volatile type of memory, e.g., flash media, disk media, etc. In various aspects of the disclosure, the processor 220 may be another type of processor, such as a digital signal processor, a microprocessor, an ASIC, a graphics processing unit (GPU), a field-programmable gate array (FPGA), or a central processing unit (CPU). In certain aspects of the disclosure, network inference may also be accomplished in systems that have weights implemented as memristors, chemically, or other inference calculations, as opposed to processors.

In aspects of the disclosure, the memory 230 can be random access memory, read-only memory, magnetic disk memory, solid-state memory, optical disc memory, and/or another type of memory. In some aspects of the disclosure, the memory 230 can be separate from the controller 200 and can communicate with the processor 220 through communication buses of a circuit board and/or through communication cables such as serial ATA cables or other types of cables. The memory 230 includes computer-readable instructions that are executable by the processor 220 to operate the controller 200. In other aspects of the disclosure, the controller 200 may include a network interface 240 to communicate with other computers or to a server. A storage device 210 may be used for storing data. The disclosed method may run on the controller 200 or on a user device, including, for example, on a mobile device, an IoT device, or a server system.

Compared to endoscopy or stool collection, the system 300 of FIG. 1 requires minimal user intervention. It operates passively with no input from the user. To achieve this, the system 300 may use an accelerometer combined with temperature and humidity sensors to automatically determine whether the system 300 is being worn. In an example, using Bluetooth™ and a custom-designed smartphone application, the system 300 can connect to a user's mobile device to upload or otherwise provide data that can be further shared with clinicians in real-time or near real-time. The Smart Underwear smartphone application could also prompt users to wear the system 300 and provide feedback about adherence to the observational study protocols. The battery life of the system 300 is more than ten days, which is longer than the proposed wearing duration for diagnosing whether the patient has a gastrointestinal disorder. Therefore, the user will not need to charge the system 300 or change batteries during the diagnosis period. The system 300 may use hearing aid batteries that are not flammable and are already certified for use in human-worn devices.

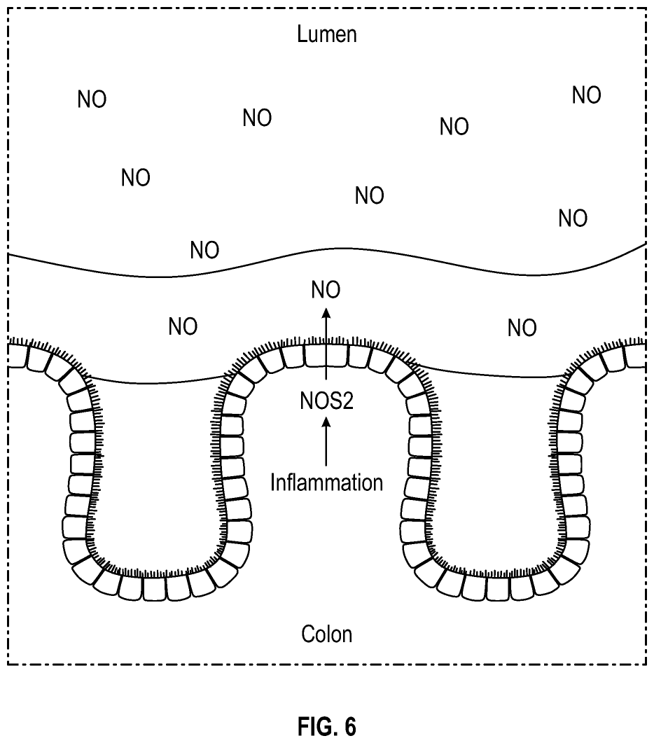

FIG. 6 illustrates the expression of the inducible nitric oxide (NO) synthase gene NOS2. NOS2 has been reported to be upregulated more than 12-fold in the rectum of patients with active ulcerative colitis. The expression of NOS2 leads to the production of NO, which can readily diffuse into the colonic lumen, where it can be expelled in flatus.

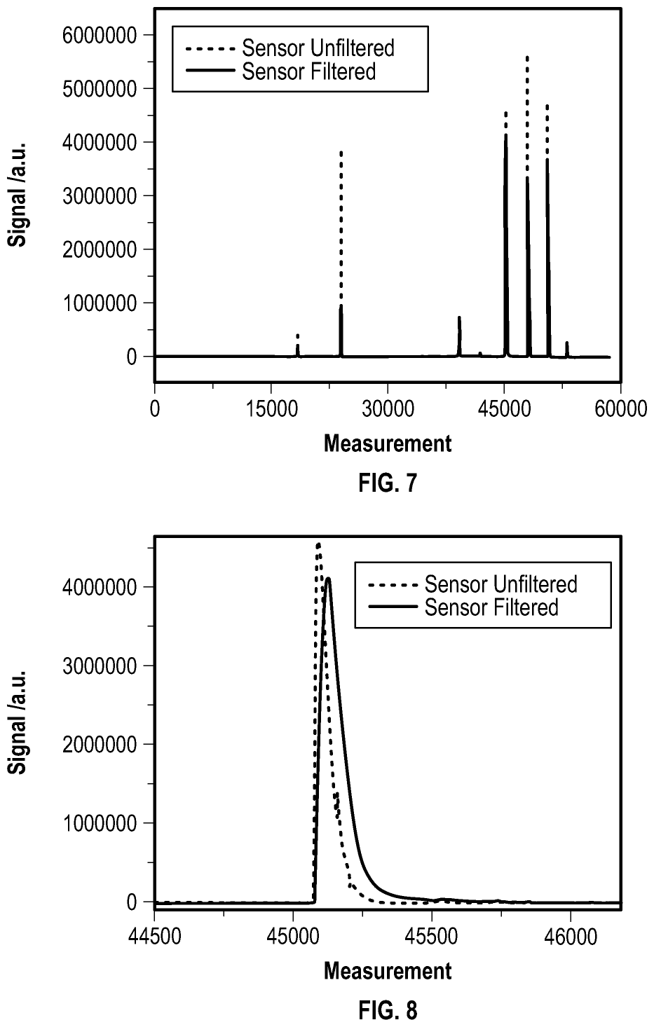

FIG. 7 illustrates data from a human wearing the Smart Underwear device for about 4 hours. Each peak corresponds with a validated flatus. The intensity of the signal is correlated with the perceived size of the flatus.

FIG. 8 illustrates a zoomed-in view of a single flatus from FIG. 7. The difference in the intensity of the signal from the unfiltered and filtered is used to calculate the concentration of H₂S present in the flatus. FIG. 9 is a graph illustrating a concentration of compounds in flatus. FIG. 10 is a graph illustrating a concentration of rectal NO in flatus based on IBD status.

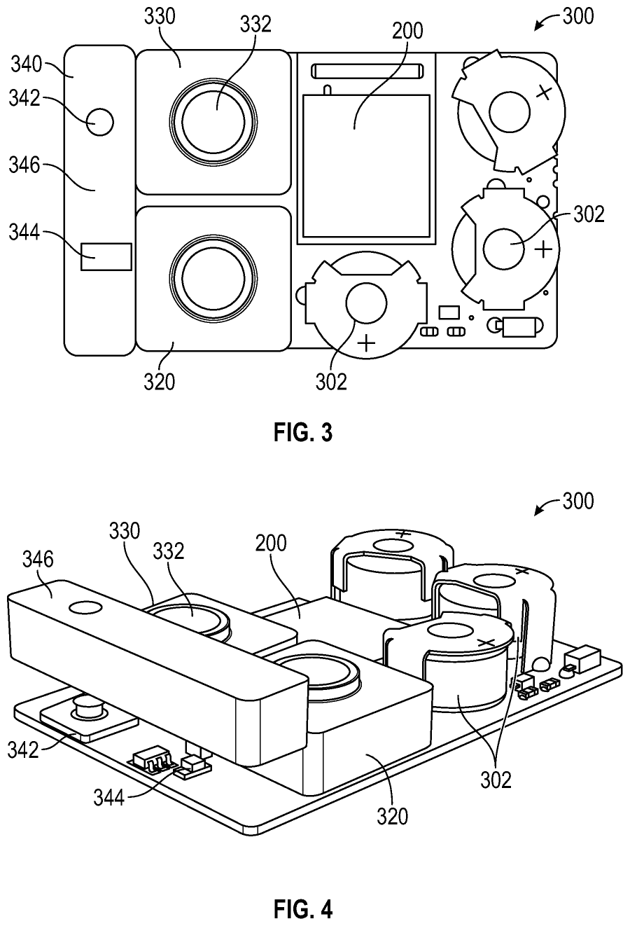

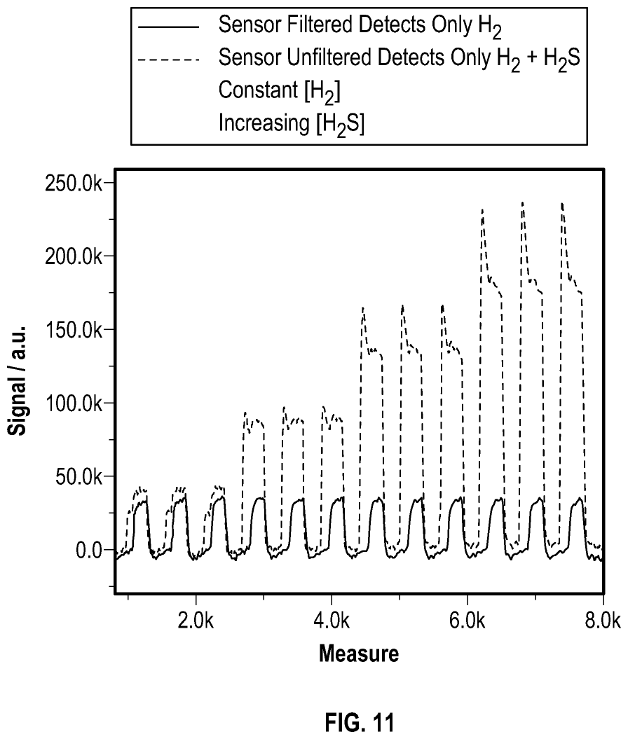

FIG. 11 is a graph that shows a comparison between the filtered sensor 330 and the unfiltered sensor 320 with increasing additions of H₂S and a fixed concentration of H₂ using a flatus simulator (not shown). The graph shows that the filter 332 of FIG. 3 removes about 98% of H₂S, and therefore, the filtered sensor signal remains constant while the unfiltered sensor measures increasing concentrations of H₂S. The flatus simulator is a gas dispenser (not shown). This custom-made device was able to reproducibly approximate the gas release of human flatus while enabling the calibration of the sensors and providing a reliable point of reference for comparison and calibration. The flatus simulator has three calibration tanks (NIST-certified), each with its own mass flow regulator, which is activated by three independent, high torque, precision servos, all controlled by a microcontroller. Gases flow through a sealed measured chamber containing the sensors or devices. The flatus simulator enables the evaluation of the signal response to different concentrations and mixtures of gases that can approximate the gas ratios reported in human flatus. The automated nature of the device has enabled reproducible measurement of hundreds of different concentrations and combinations of gases to rationally choose the best sensors and filtration strategies. In addition, the flatus simulator enables the benchmarking of newly fabricated devices and filters.

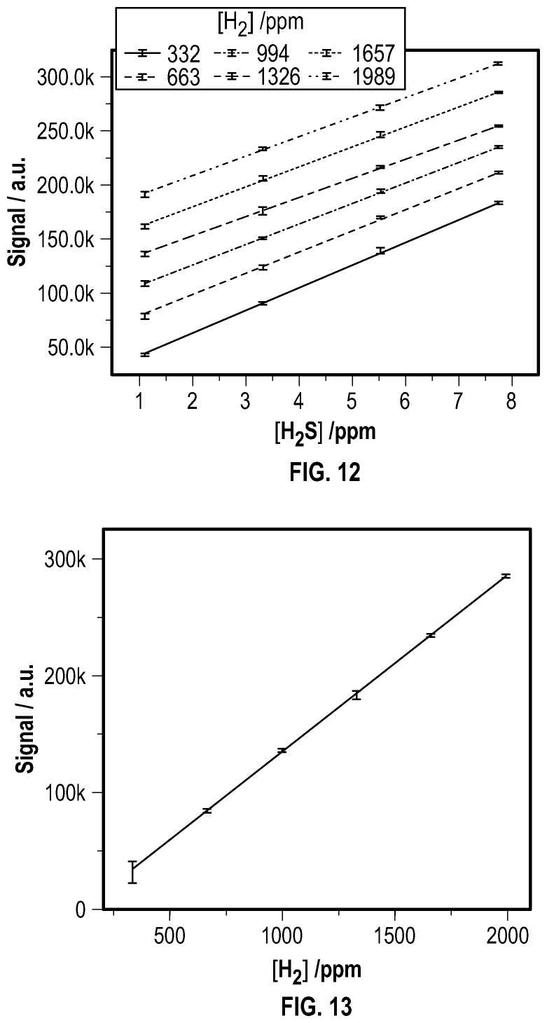

FIG. 12 illustrates calibration curves from the unfiltered sensor by keeping the concentration of H₂ constant for each curve and increasing only the concentration of H₂S, using the flatus simulator. The results show that all curves are parallel, meaning that no synergistic effect was observed due to the gas mixture, and therefore, the sensor response is additive.

FIG. 13 is a graph that shows a calibration curve for hydrogen concentration using the reading from the filtered sensor 330 of FIG. 3.

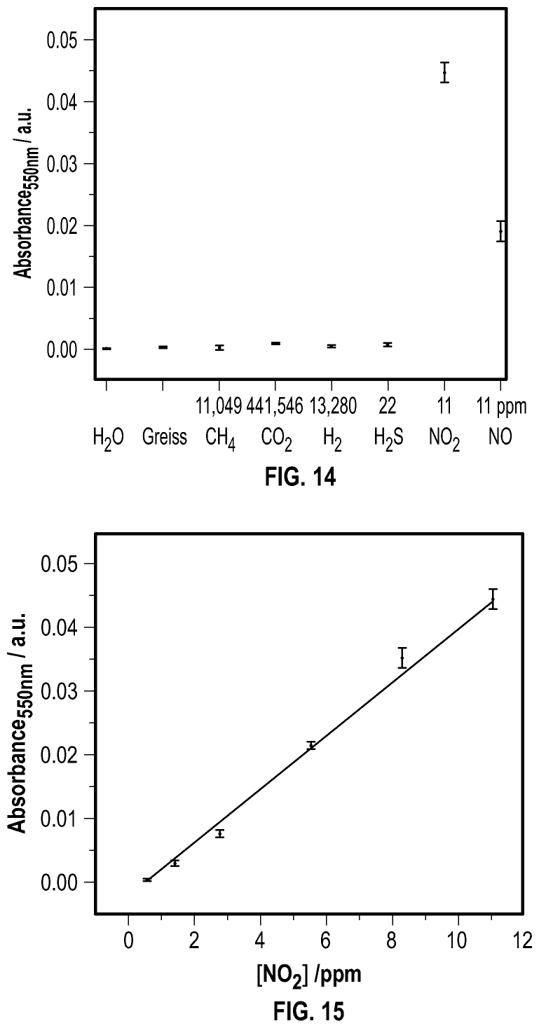

FIG. 14 is a graph that illustrates NO₂ detection using the Griess reaction. The signal increase in the absorbance is observed with the increase in the NO₂ gas concentration using a flatus simulator. For each measure, the concentration was kept constant for 5 minutes. The proof-of-concept for the NO detection in flatus was demonstrated using the flatus simulator. Concentrations above 2.8 ppm of NO₂ can be unambiguously detected after incubating 100 uL Griess solution (1/5 X) in the measurement chamber for 5 min (FIG. 15). FIG. 15 is a graph that illustrates a linear profile with an increasing concentration of NO₂ using a flatus simulator. No interference from other components of flatus (H₂O, H₂, H₂S, CH₄) was noted, consistent with the chemical specificity of the Griess reaction.

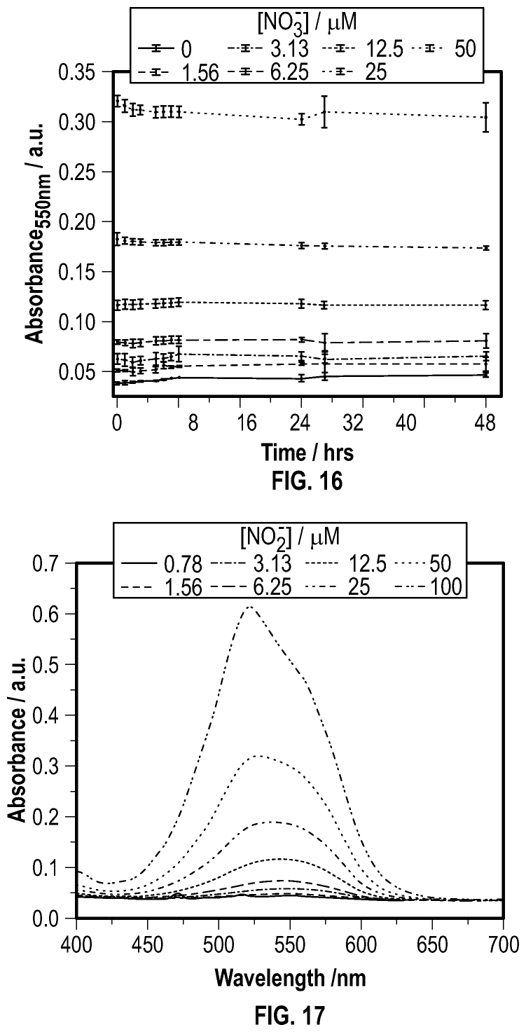

FIG. 16 is a graph that illustrates the stability of the Griess solution. The reagent is stable for at least two days at all concentrations. The optical path is about 0.1 cm. Unlike other sensitive quantification techniques, such as chemiluminescence, the colorimetric reaction with the Griess reagent is stable for at least 48 hours, giving enough time for the concentration of NO and its derivatives to result in a change in color of the Griess reagent if the subject excretes 15-20 flatus during that time. The color change was measured with a benchtop spectrophotometer, but the detection method can be replaced by the spectroscopic sensor 340 (FIG. 19).

FIG. 17 is a graph that illustrates a spectra profile of Griess reaction with nitrite. The graph shows a maximum of around 550 nm. The light sensor was evaluated using serial dilutions of standard nitrite (NO₂) incubated for half an hour with 20 μL of Griess reagent. The results are summarized in FIG. 11, which shows a linear regression (R²=0.99) between 0-8 μM. The sensor's green channel (550 nm) was used as a measure of the light intensity, and therefore the “equivalent” absorbance was calculated:

$$absorbance = - log T$$

$$T = \frac{I}{{I}_{0}}$$

$$absorbance = - log \frac{{Signal}_{550 nm}}{{Signal}_{550 nm , 0}}$$

where T is the transmittance defined by the quotient between the intensity of light that reaches the sensor divided by the intensity emitted from the light source

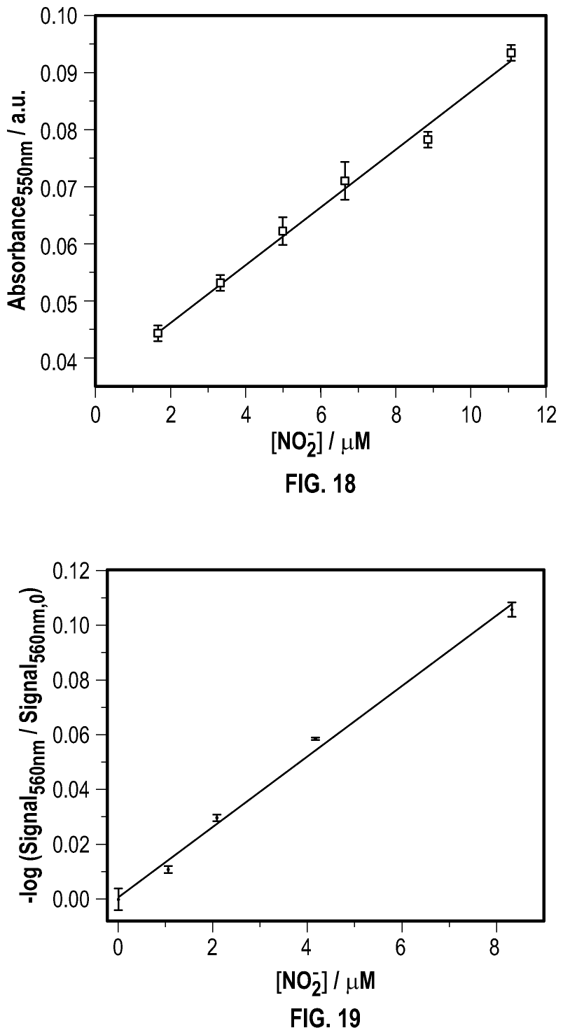

FIG. 18 is a graph that illustrates a calibration curve using the spectrophotometer and Griess reagent with an increasing concentration of nitrite.

FIG. 19 is a graph that illustrates a calibration curve with the spectroscopic sensor 340 of FIGS. 3 and 100 μL of Griess reagent with Increasing additions of nitrite. The raw signal was used as a measure of the light intensity, and the value was used with the definition of the absorbance showing a linear plot.

Inflammation impairs the absorptive capacity of the intestines resulting in excess amino acids escaping human absorption and being fermented by the human gut microbiota, which produces H₂S as the byproduct. Testing for gastrointestinal inflammation is extremely common in clinical practice, but stool biomarkers, such as calprotectin and lactoferrin are inaccurate, and endoscopy is expensive and highly invasive. Changes in H₂S production that are indicative of gastrointestinal inflammation could be detected by smart underwear and be an important clinical tool for diagnosing inflammation.

There is demand from pharmaceutical companies for tools that can enable the evaluation of the efficacy of the new anti-inflammatory drugs in the context of Inflammatory Bowel Disease (Crohn's disease and Ulcerative Colitis). Using smart underwear to monitor H₂S production over the course of weeks could dramatically increase the temporal resolution of clinical trials measuring the efficacy of these drugs.

In aspects, the disclosed systems and methods may be used for implementing precision nutrition strategies. Excess dietary amino acids that are not absorbed by the host are fermented by the human gut microbiota leading to harmful byproducts which are implicated in the etiology of numerous diseases, including obesity and Type 2 Diabetes. There is a large inter-individual variation in the capacity to absorb amino acids. Therefore, strategies to assess amino acid absorption are necessary to tailor dietary intake. One of the byproducts of excess sulphur-containing amino acid consumption is H₂S, which can be measured by the Smart Underwear device. Therefore, Smart Underwear could be used by both dieticians and members of the public to determine the optimal amount of dietary amino acids based on the quantity that can be absorbed by the host. In addition, the Smart Underwear device could be easily modified to measure many other clinically-relevant byproducts of gut microbial metabolism.

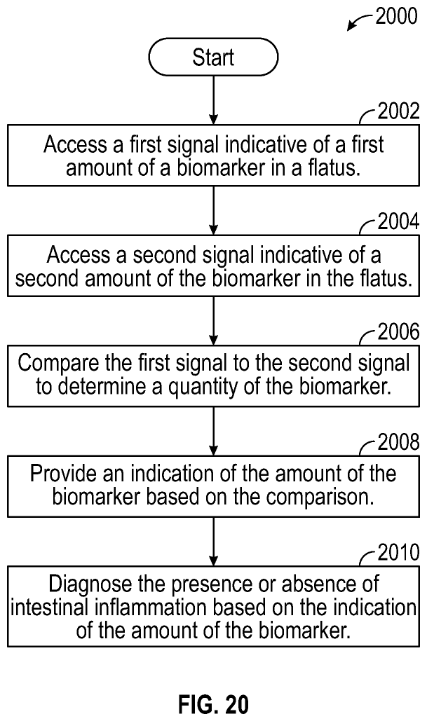

Referring to FIG. 20, a flow diagram for a method in accordance with the present disclosure for diagnosing the presence or absence of intestinal inflammation is shown as 500. Although the steps of FIG. 20 are shown in a particular order, the steps need not all be performed in the specified order, and certain steps can be performed in another order. For example, FIG. 20 will be described below, with a controller 200 of FIG. 2 performing the operations. In aspects, the operations of FIG. 20 may be performed all or in part by another device, for example, a mobile device, such as a smartphone, and/or a computer system. These variations are contemplated to be within the scope of the present disclosure.

Initially, at step 2002, the controller 200 accesses a first signal indicative of a first concentration of a biomarker in a flatus. The first signal may be generated by a first sensor 320 of the system 300 of FIG. 3. For example, the first sensor 320 may be an electrochemical (e.g., amperometry) sensor. The sensor 320 may measure, for example, hydrogen, hydrogen sulfide, methane, nitric oxide, and/or nitrogen dioxide.

Next, at step 2004, the controller 200 accesses a second signal indicative of a second concentration of the biomarker in the flatus. The second signal may be generated by a second sensor 330. For example, the first sensor may be an electrochemical sensor. The second sensor 330 may include a filter 332 configured to selectively filter a volatile compound such as H₂S. The first sensor 320 and second sensor 330 may be the same type of sensor. For example, the controller 200 may measure a concentration of nitric oxide and/or nitrogen dioxide in a flatus. In aspects, each sensor prior to use may be calibrated, and the calibration may be stored on the controller 200. The calibration may be used to zero out each of the sensors to ambient gas. The second concentration may include none of the biomarker, for example in the case where the filter completely removes all of the biomarker.

In aspects, the first sensor 320 and second sensor 330 may be part of a system 300 that is adhered to a user's undergarment (FIG. 1). For example, the system 300 may be positioned on the outside of a user's underwear adjacent to the anus attached via double-sided tape. Therefore, the system 300 does not contact a user's skin.

Next, at step 2006, the controller 200 compares the first signal to the second signal to determine a concentration of the biomarker.

In aspects, the controller 200 may use a background-subtraction approach for measuring hydrogen sulfide, nitric oxide, and nitrogen dioxide in a high hydrogen background. In aspects, the controller may compare the measured concentration of the biomarker against an expected concentration of the biomarker in a flatus of a healthy user. In aspects, the controller 200 may use a Griess reaction measured by a spectroscopic sensor to detect nitric oxide and nitrogen dioxide. In aspects, the controller 200 may access a signal from a spectroscopic sensor 340 for sensing a color change indicating NO and/or NO₂ detection. In aspects, the controller 200 may detect a color change in a Griess reagent of the spectroscopic sensor 340 and determine a concentration of the biomarker, based on the color change.

Next, at step 2008, the controller 200 provides an indication of the concentration of the biomarker, based on the comparison.

Next, at step 2010, the controller 200 diagnoses the presence or absence of intestinal inflammation and/or an Inflammatory Bowel Disease based on the indication of the concentration of the biomarker. Inflammatory Bowel Disease may include Crohn's disease, ulcerative colitis, and indeterminate colitis. In aspects, the various sensor signals may be used as inputs to a machine learning network (e.g., a convolutional neural network), which may predict the presence or absence of an intestinal disorder based on the sensor signals. The machine learning network may be trained on prior clinical data.

The controller 200 may use gut microbial gas production as a proxy for gut microbiome activity. In aspects, the system 300 of FIG. 1 may be used to diagnose the presence or absence of small intestine bacterial overgrowth (SIBO) by the measurement of gut microbial hydrogen in flatus. Initially, the controller 200 may measure a concentration of hydrogen, methane, and/or hydrogen sulfide in flatus. The controller 200 may sum the concentrations of all measured flatus into a daily production of the aforementioned gases. The controller 200 may compare the daily production of the aforementioned gases against expected concentrations in healthy individuals. The controller 200 may diagnose the presence or absence of SIBO based on the comparison.

In aspects, the disclosed systems and methods may be used to assess the efficacy of probiotic or prebiotic interventions and/or may be used to screen for side effects of probiotic or prebiotic interventions.

In aspects, the disclosed systems and methods may be used to measure increased NO production and/or increased gut microbial H₂S production during UC flares.

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an aspect,” “in aspects,” “in various aspects,” “in some aspects,” or “in other aspects” may each refer to one or more of the same or different example Aspects provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A system for detecting and/or managing a gastrointestinal disorder, the system comprising:
a first sensor configured to generate a first signal indicative of sensing of a first concentration of a biomarker in a flatus, the flatus including a mixture of gases;
a second sensor configured to generate a second signal indicative of sensing of a second concentration of the biomarker in the flatus;
a filter disposed on the second sensor, the filter configured to selectively remove the biomarker from the flatus prior to sensing by the second sensor;
a processor; and
a memory including instructions stored thereon, which, when executed by the processor, cause the system to:
compare the first signal to the second signal to determine a concentration of the biomarker; and
provide an indication of the concentration of the biomarker, based on the comparison.

2. The system of claim 1, further comprising a third sensor, the third sensor is configured to generate a third signal indicative of sensing of a temperature or a humidity.

3. The system of claim 2, wherein the instructions, when executed by the processor, further cause the system to compensate the first signal and the second signal based on the third signal.

4. The system of claim 1, wherein the first sensor and the second sensor are the same type of sensor.

5. The system of claim 1, wherein the first sensor and the second sensor include at least one of an electrochemical sensor and/or a metal oxide sensor.

6. The system of claim 1, further comprising an accelerometer configured to detect motion.

7. The system of claim 6, wherein the instructions, when executed by the processor, further cause the system to determine whether the system is being worn by a user based on the detected motion.

8. The system of claim 1, further comprising a spectroscopic sensor configured for sensing a color change indicating biomarker detection.

9. The system of claim 6, wherein the instructions, when executed by the processor, further cause the system to:
detect a color change in a Griess reagent; and
determine a concentration of the biomarker, based on the color change.

10. The system of claim 1, wherein the instructions, when executed by the processor, further cause the system to diagnose a presence, an absence, and/or an activity of intestinal inflammation based on the indication of the concentration of the biomarker.

11. A computer-implemented method for detecting and/or managing a gastrointestinal disorder, the method comprising:
accessing a first signal indicative of a first concentration of a biomarker in a flatus;
accessing a second signal indicative of a second concentration of the biomarker in the flatus;
comparing the first signal to the second signal to determine a concentration of the biomarker; and
providing an indication of the concentration of the biomarker, based on the comparison.

12. The computer-implemented method of claim 11, further comprising diagnosing a presence or absence of intestinal inflammation based on the indication of the concentration of the biomarker in the flatus.

13. The computer-implemented method of claim 11, wherein the first signal is sensed by a first sensor and the second signal is sensed by a second sensor.

14. The computer-implemented method of claim 13, wherein the first sensor and the second sensor are the same type of sensor.

15. The computer-implemented method of claim 13, further comprising selectively removing the biomarker from the flatus prior to sensing by the second sensor by a filter disposed on the second sensor.

16. The computer-implemented method of claim 13, further comprising:
sensing by a third sensor a third signal indicative of a temperature or a humidity; and
determining whether a system that includes the first sensor, the second sensor, and the third sensor is being worn by a user, based on the third signal.

17. The computer-implemented method of claim 13, further comprising:
detecting motion by an accelerometer; and
determining whether a system that includes the first sensor, the second sensor, and the accelerometer is being worn by a user, based on the detected motion.

18. The computer-implemented method of claim 11, further comprising sensing a color change of a Griess reagent disposed in a flatus by a spectroscopic sensor, wherein the color change indicates biomarker detection.

19. The computer-implemented method of claim 18, further comprising determining a concentration of the biomarker, based on the color change.

20. A smart underwear system for detecting and/or managing a gastrointestinal disorder, the system including:
an undergarment configured to be worn by a user; and
a device attached to the undergarment and configured to diagnose a presence, absence, and/or activity of the gastrointestinal disorder, the device comprising:
a first sensor configured to generate a first signal indicative of a first concentration of a biomarker in a flatus;
a second sensor configured to generate a second signal indicative of a second concentration of the biomarker in the flatus;
a filter disposed on the second sensor, the filter configured to selectively remove the biomarker from the flatus prior to sensing by the second sensor;
a processor; and
a memory including instructions stored thereon, which, when executed by the processor, cause the device to:
compare the first signal to the second signal to determine a concentration of the biomarker; and
provide an indication of the concentration of the biomarker, based on the comparison.
